(19) United States
(12) Patent Application Publication
(10) Pub. No.: US 2014/0273090 A1
(43) Pub. Date: Sep. 18, 2014

(54) Methods and Systems for Peptide/Protein Amplification

(71) Applicant: Arizona Board of Regents, a body corporate of the State of Arizona, acting for and on behalf of, Scottsdale, AZ (US)

(72) Inventor: Rolf U. Halden, Phoenix, AZ (US)

(73) Assignee: Arizona Board of Regents, a body corporate of the State of Arizona, acting for and on behalf of, Scottsdale, AZ (US)

(21) Appl. No.: 14/207,003

(22) Filed: Mar. 12, 2014

Related U.S. Application Data
(60) Provisional application No. 61/779,822, filed on Mar. 13, 2013.

(51) Int. Cl. C12P 21/00; C12P 19/34

(52) U.S. Cl. CPC C12P 21/00; C12P 19/34
USPC 435/69.1; 435/91.2

(57) ABSTRACT

An amino acid chain of interest is selected or captured for amplification. The amino acid chain of interest including at least one amino acid sequence is denatured and read. The read amino acid sequence is transcribed to synthesize a mRNA equivalent. The amino acid chain of interest is then amplified using a biological system.

### RELATED APPLICATION

This application claims priority from and is a continuation-in-part of co-pending U.S. provisional application No. 61/779,822 of Halden, filed Mar. 13, 2013, entitled “Methods and Systems for Protein Amplification.” U.S. provisional application No. 61/779,822 is hereby incorporated by reference.

### TECHNICAL FIELD

The present invention relates to methods and systems for protein amplification. More particularly, the present invention relates to methods and systems for the determination of peptide sequences and subsequent mass production of peptides/proteins using inexpensive cellular or in vitro processes for peptide/protein production and modification.

### BACKGROUND

Biotechnology relies heavily on the use of peptides and proteins serving diverse functions. Whereas the Polymerase Chain Reaction (PCR) enables the amplification of DNA to large quantities of DNA copies, a similar convenient biological copy machine for peptides and proteins is not known.

In an advance over the state of the art, the present invention provides a solution to the long felt need for amplifying peptides and proteins. Methods and systems are disclosed enabling those skilled in the arts to rapidly, inexpensively and conveniently create as many copies of a target peptide or protein as desired using a peptide/protein amplification reaction (PAR) method from one or more protein/peptide templates.

### SUMMARY OF THE DISCLOSURE

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

A peptide/protein amplification reaction (PAR) method includes selecting or capturing an amino acid chain of interest for amplification. The amino acid chain of interest including at least one amino acid sequence is denatured. The at least one amino acid sequence and any protein modifications present is read. The method further includes synthesizing at least one mRNA template, mRNA equivalent, or cDNA template complementary to the read amino acid sequence; and translating the mRNA, mRNA equivalent, or cDNA to obtain an amplified amino acid chain of interest using a biological system.

In one aspect the amino acid chain of interest comprises a peptide or protein.

In another aspect the biological system comprises a ribosome driven system.

In another aspect the step of reading the at least one amino acid sequence and applicable protein modifications comprises processing the at least one amino acid sequence by operating a scanning tunneling microscope and/or an atomic force microscope.

In another aspect the step of reading the at least one amino acid sequence and applicable protein modifications comprises processing the at least one amino acid sequence by performing de novo sequencing using mass spectrometry.

In another aspect the step of synthesizing at least one mRNA template, mRNA equivalent, or cDNA template includes transcribing the read amino acid sequence to synthesize a mRNA, mRNA equivalent, or cDNA using oligonucleotide synthesis.

In another aspect the method further comprises increasing the number of mRNA templates, mRNA equivalents, or cDNA.

In another aspect the step of increasing the number of mRNA equivalent templates comprises processing the mRNA template, mRNA equivalent, or cDNA template by nucleic acid amplification (PCR).

In another aspect the step of increasing the number of mRNA templates, mRNA equivalents, or cDNA comprises processing the mRNA template, mRNA equivalent, or cDNA by RT-PCR.

In another aspect the method further comprises modifying the amplified amino acid chain of interest.

In another aspect the step of to obtain the amplified amino acid chain of interest using a biological system is carried out in vivo.

In another aspect the step to obtain the amplified amino acid chain of interest using a biological system is carried out in vitro.

In another aspect the step to obtain the amplified amino acid chain of interest using a biological system is carried out on a solid surface.

In another aspect the step to obtain the amplified amino acid chain of interest using a biological system is carried out on a solid surface with immobilized protein.

Other benefits and advantages of the present invention will become apparent from the disclosure, claims and drawings herein.

### BRIEF DESCRIPTION OF THE DRAWINGS

While the novel features of the invention are set forth with particularity in the appended claims, the invention, both as to organization and content, will be better understood and appreciated, along with other objects and features thereof, from the following detailed description taken in conjunction with the drawings, in which:

FIG. 1 schematically shows a representation of protein synthesis.

FIG. 2 schematically shows one example of a high level process flow for one example of a protein amplification reaction (PAR) method.

FIG. 3 schematically shows a process for reading amino acid sequences as employed in one example of a PAR method.

FIG. 4 schematically shows a process for synthesizing amino acid sequences as employed in one example of a PAR method.

FIG. 5 schematically shows examples of synthetic mRNA configurations as employed in one example of a PAR method.

FIG. 6 schematically shows final stages of process flow as employed in one example of a PAR method.

FIG. 7 schematically shows steps involved in cell-free production of peptides/proteins in one example of a PAR method.

FIG. 8 schematically shows steps involved in the cell-free production of peptides/proteins on a solid surface in one example of a PAR method.

FIG. 9 schematically shows a detailed diagram of steps involved in the cell-free production of peptides/proteins on a solid surface in one example of a PAR method is schematically shown to further understanding of the method described with respect to FIG. 8.

In the drawings, identical reference numbers identify similar elements or components. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn, are not intended to convey any information regarding the actual shape of the particular elements, and have been solely selected for ease of recognition in the drawings.

### DETAILED DESCRIPTION OF THE PREFERRED EMBODIMENTS

The following disclosure describes several methods and systems for protein and peptide amplification. Several features of methods and systems in accordance with example embodiments are set forth and described in the Figures. It will be appreciated that methods and systems in accordance with other example embodiments can include additional procedures or features different than those shown in the Figures. Example embodiments are described herein with respect to amino acid chains. However, it will be understood that these examples are for the purpose of illustrating the principles, and that the invention is not so limited.

Additionally, methods and systems in accordance with several example embodiments may not include all of the features shown in these Figures. Throughout the Figures, identical reference numbers refer to similar or identical components or procedures.

Unless the context requires otherwise, throughout the specification and claims which follow, the word “comprise” and variations thereof, such as, “comprises” and “comprising” are to be construed in an open, inclusive sense that is as “including, but not limited to.”

Reference throughout this specification to “one example” or “an example embodiment,” “one embodiment,” “an embodiment” or various combinations or variations of these terms means that a particular feature, structure or characteristic described in connection with the embodiment is included in at least one embodiment of the present disclosure. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

### DEFINITIONS

Generally, as used herein, the following terms have the following meanings:

“DNA” is used in its usual sense and means deoxyribonucleic acid.

“RNA” is used in its usual sense and means ribonucleic acid.

“mRNA” is used in its usual sense and means messenger RNA, a codon.

“tRNA” is used in its usual sense and means transfer RNA, an anti-codon.

“PAR” as used herein relates to Peptide/protein Amplification Reaction using the methods and systems disclosed herein.

“PCR” is used in its usual sense to mean Polymerase Chain Reaction which is a well-known biochemical technology for amplifying one or more pieces of DNA to generate thousands of copies of a particular DNA sequence.

“RT-PCR” is used in its usual sense to mean Reverse Transcription Polymerase Chain Reaction.

### INTRODUCTION

The non-obvious and novel combination of various established techniques in biotechnology disclosed herein can produce methods and systems enabling the unlimited amplification of target peptides/proteins from a small number of templates where the number of templates is equal to or greater than 1. Cells, cell extracts and their components constitute a convenient biological pipeline for production of peptides/proteins. To arrive at a protein amplification reaction (PAR), one needs to read the amino acid sequence of a peptide/protein of interest and translate it into a mRNA, “mRNA equivalent” or cDNA that can be synthesized chemically or biochemically and then fed into the existing biological pipeline for peptide/protein production.

For the sake of introduction, generally the herein-disclosed method is exemplified by a combination of the following steps:

1) Selecting or capturing a protein template such as a peptide or protein of interest for amplification;

2) Unfolding and linearization of the amino acid sequence of interest using established techniques for protein denaturing;

3) Reading of the amino acid sequence by using established techniques for observing DNA sequences. Such techniques may include, for example, N-terminal or C-terminal sequencing, mass spectrometry, and tunneling sequencing approaches. For example, scanning tunneling microscopes (STM) and atomic force (ATM) microscopes (ATM) may be used to “read” amino acid sequences (e.g., as by pulling through a thread and reading off amino acid sequences). The latter approach was first developed for inexpensive DNA sequencing. A selection of suitable techniques is shown in FIG. 3. Such methods typically yield a unique electrical signal for each of 20 common amino acids and also can serve to detect and deduce post-translational modifications on target peptides or proteins.

4) Transcribing the “read” amino acid to automatically synthesize a mRNA, mRNA equivalent or cDNA, which can serve as a template for protein amplification.

5) Optionally amplifying the mRNA equivalent or cDNA using PCR.

6) Massive translating of the mRNA equivalent or cDNA in parallel using inexpensive cell systems, cell extracts, and/or cell extract components, or other suitable techniques practiced by those skilled in the art.

7) Optionally performing protein folding or modification to create native, functional protein and desired post-translational modifications.

The aforesaid steps thereby enable amplification of peptides/proteins from few or, in the extreme, from a single template to large quantities. In addition, the use of tunnel recognition and other methods (such as de novo sequencing by tandem mass spectrometry) for sequence determination enable the “reading” of protein modifications along with the primary sequence of amino acids. The detected modifications later are reproduced with high fidelity in the amplified peptides/proteins by exploiting targeted post-expression modification of peptides and proteins present free in solution or immobilized on a solid-state surface.

### DETAILED DISCUSSION

Referring now to FIG. 1 a representation of protein synthesis is schematically shown. mRNA or a mRNA equivalent is needed to start the protein synthesis. In one useful example, protein synthesis requires operation of one or more ribosomes 1 on a strand of mRNA 3. Protein molecules 5 are formed when a mRNA codon 7 or series of codons are combined by one or more ribosomes 1 with the help of anti-codon transfer RNA (tRNA) 9 which binds with amino acids 11 before mating with its matching mRNA codon.

Referring now to FIG. 2 one example of a high level process flow for one example of a protein amplification reaction (PAR) method is schematically shown. The process begins with selecting or capturing a protein template of interest for amplification 20. The protein of interest is denatured or linearized using established techniques 22. In a next step 24 reading of an amino acid sequence is implemented using established techniques for observing DNA sequences. For example, microscopy employing scanning tunneling (STM) and atomic force (ATM), may advantageously be used to “read” amino acid sequences and peptide/protein modifications, e.g., as by pulling through a thread and reading off an amino acid sequence¹.

Oligonucleotide synthesis 26 is then implemented for transcribing the “read” amino acid to automatically synthesize a “mRNA” equivalent or cDNA, which can serve as a template for protein amplification. Another step includes increasing the number of mRNA equivalent templates at will, by using nucleic acid amplification (PCR) 30. mRNA equivalents suitable for this reaction step have to fulfill the requirement of mimicking DNA that directly can enter into the PCR or mimicking RNA that can be reverse transcribed before entering the PCR. As shown in FIG. 4, established biological system(s) may be used to amplify peptide/protein of interest to the desired quantity using the mRNA equivalent as a template 32. An optional step 34 for protein folding or modification may be included to create native, functional protein mass.

The above-described process enables amplification of peptides/proteins from few or, in the extreme, from a single template to large quantities and does produce products of a fidelity honoring both amino acid sequence and post-translational modifications.

Referring now to FIG. 3, a process for reading amino acid sequences 300 as employed in one example of a PAR method is schematically shown. The process of “reading” may advantageously include operating a mass spectrometer for de novo peptide sequencing, performing N-terminal or C-terminal sequencing, for example, by using phenyl isothiocyanate (PITC) as a labeling agent, using C-terminal sequencing, employing Hierarchical Linear and Nonlinear Modeling (HLM), or using recognition tunneling and atomic force microscopy as is known by those skilled in the art. If the number of template peptides/proteins is equal to or larger than one and if a sufficiently sensitive mass spectrometer is available, then the sequence of amino acids and any given modifications on the peptide/protein also can be determined by de novo sequencing using state of the art tandem mass spectrometry².

Referring now to FIG. 4, a process for synthesizing amino acid sequences as employed in one example of a PAR method is schematically shown. Shown is a commercially available synthesizer 50 that produces a mRNA strand or mRNA strand equivalent 52. Alternatively, synthesizer 50 can directly produce a complementary DNA (cDNA) 63 complementary to the amino acid sequence read.

Referring now to FIG. 5, examples of synthetic mRNA configurations as employed in one example of a PAR method are schematically shown. A first example of a synthetic mRNA or mRNA equivalent configuration 55 and a second example of a synthetic mRNA 57 are generated and are suitable for use in the PAR process. The cDNA complementary to the amino acid sequence 63 (as shown in FIG. 6) can also be produced directly.

Now referring to FIG. 6, final stages of the process flow as employed in one example of a PAR method are schematically shown. There is a plurality of methods for arriving at the desired peptide/protein amplification depending upon the mRNA or mRNA equivalent synthesized. In a first exemplary method following process 60 the mRNA is synthesized using a commercially available synthesizer 50 or the like producing mRNA strand 52. mRNA strand 52 is then used as a template for RT-PCR to create multiple copies of complementary DNA (cDNA) 63. Alternatively, the automatic synthesizer may be used to directly obtained cDNA. The multiple copies of cDNA are then combined in a ribosome driven process within a cell 65 to produce multiple proteins or peptides 70. Optional protein manipulation 34 may be carried out to fold the peptides/proteins into desired two and three-dimensional configurations 80.

In a second exemplary method following the in vitro process 200 a first synthetic mRNA strand 55 and a second synthetic mRNA strand 57 are introduced into a test tube 77. They may optionally be amplified using PCR into multiple synthetic mRNA equivalent strands. The synthetic mRNA strands or amplified mRNA equivalent strands are then used as a template combined in a biological system such as a ribosome driven process to produce multiple proteins or peptides 102. Optional protein manipulation may be carried out as before to fold the peptides/proteins 70′ into folded configurations 80.

Referring now to FIG. 7, steps involved in cell-free production of peptides/proteins in one alternative example of a PAR method carried out in vitro on a solid surface is schematically shown. The entire process of protein assembly and modification can be carried out in vitro on a solid surface (protein platform) by leveraging downstream approaches, such as self-assembling protein microarrays (as described by LaBaer and Ramachandran¹²). When leveraging this approach, protein amplification begins with the previously described protein sequence determination, for example, by tunnel recognition methods 24. Next is the synthesis of a coding region corresponding to the peptide sequence obtained by peptide/protein sequence determination. The corresponding gene sequence is synthetically obtained. In one approach, a set of individually designed oligonucleotides may be made on automated solid-phase synthesizers, purified and then connected by specific annealing and standard ligation or polymerase reactions 26. To improve specificity of oligonucleotide annealing, the synthesis step may rely on the use of thermostable DNA ligase and polymerase enzymes. Suitable methods for gene synthesis include but are not limited to the ligation of phosphorylated overlapping oligonucleotides^{3,4} the Fok I method⁵ and a modified form of ligase chain reaction for gene synthesis. Additionally, several PCR assembly approaches 30 have been described⁶. These may employ oligonucleotides that overlap. Overlapping oligonucleotides may be designed to cover most of the sequence of both strands; the desired molecule of full length is then obtained progressively by overlap extension (OE) PCR⁶ thermodynamically balanced inside-out (TBIO) PCR⁷ or combined approaches⁸ as is understood by those skilled in the art⁹.

The PAR can yield mRNA or a mRNA equivalent as an intermediate product. However, it may also bypass the synthesis of mRNA and instead directly produce by automatic synthesis or other means a cDNA complementary to the aminoacid sequence of interest. The teachings of Ramachandran et al (2004) provide the methodology of producing a desired protein directly from synthetic cDNA in a cell free biological system.

On a side note, computer-assisted selection of the most suitable codons can aid in improving the yield of proteins. Protein expression in both in vitro and in vivo (e.g., bacterial cells) can be maximized by preferential use of codons for tRNA's which retain amino acid charging during starvation¹⁰. This process enabled by appropriate computer programs¹¹ may favorably optimized a gene to improve protein expression by up to 100-fold⁹.

To avoid the reliance on intact cellular biological systems for protein expression, the resultant gene sequences can be integrated in Open Reading Frames (ORFS) which are then integrated into vectors. This approach utilizes the ability to transfer protein-encoding ORFS into customized expression vectors that are tagged. The resultant expression clones then are deposited (spotted) on an array, such as a microtiter plate surface (as illustrated in FIG. 9). The desired protein then is produced on the array surface in a cell-free system and immobilized in place upon synthesis. This approach has been described in the literature¹² as follows: “The recent development of a self-assembling protein microarray, called nucleic acid programmable protein array (NAPPA) is based on this principle¹³. In this case, full length cDNA molecules—not purified proteins—are immobilized on a microarray surface and expressed in situ using a mammalian cell-free expression system (rabbit reticulocyte lysate). A fusion tag present on the protein is recognized by a capture molecule arrayed (along with the cDNA) on the chip surface. This capture reaction then immobilizes the protein on the surface in a microarrayed format. This approach obviates the need to express, purify and store the proteins. As the proteins are freshly synthesized just-in-time for assaying, there is less concern about protein stability. This approach produces a sizable amount of protein per feature (270-2700 pg), averaging about 10 fmols. The microarrays are stable dry at room temperature until they are activated to make protein.” This approach originally was developed for the detection of protein-protein interactions. Leveraging this approach for the amplification of single or few peptides/proteins is beneficial for the PAR 700. The aforementioned approach reduces the need for extensive direct manipulations of the peptides/proteins and avoids problems with protein purification and stability. The literature describes innovative methods for creating such arrays, thereby allowing the production of one and the same or thousands of differing proteins simultaneously in vitro on the array surface; the validity and practicality of this approach has been demonstrated¹⁴. An alternative in vitro approach involves the expression of proteins using human HeLa lysate¹⁵.

An added benefit of the cell-free in vitro synthesis of proteins on an array is that they can be manipulated 702 to obtain desired post-translational modifications, such as phosphorylation, AMPylation, or citrullination. Upon completion of modification/customization, the resultant peptides/proteins are cleaved of the array 704, using, e.g., anchor-specific enzymes, and are ready for use. The array-based production of proteins assists in overcoming known limitations in protein production in biological systems. It addresses the concern of peptide/protein stability. In addition, it is less time-consuming and less costly to produce proteins of high fidelity in a sufficient quantity; it also addresses the known limitations in purifying proteins from biological systems upon protein expression. Indeed, the combination of reading the structural attributes of single molecules with emerging tools such as tunneling recognition, and then applying this knowledge to recreate as many copies of the template as desired using solid-state protein platform, create a new, innovative, non-obvious, practical and valuable functionality.

Referring now to FIG. 8, detailed steps involved in the cell-free production of peptides/proteins on a solid surface in one example of a PAR method is schematically shown to further understanding of the method described above with respect to FIG. 7. Using an in vitro system as taught by LaBaer and Ramachandran, ¹² proteins may be produced by simultaneous expression and capture of the protein on the array surface. The process includes spotting of cDNA on a solid surface 863 followed by in situ protein synthesis using an in vitro system 890 producing a surface with amplified protein of interest 892 and optionally using peptide/protein manipulation 894 before protein release LL. The optional protein manipulation 894 may be carried out to fold the peptides/proteins into desired two and three-dimensional configurations.

Referring now to FIG. 9, a detailed schematic diagram of steps involved in the cell-free production of peptides/proteins on a solid surface in one example of a PAR method is schematically shown to further understanding of the method described above with respect to FIG. 8. cDNA 864 is spotted on a solid surface 868. Fusion tags are used to hold a target protein at a distance from the matrix, exposing more overall surface area to solvent, but sterically blocking either the N- or C-terminus and requiring the addition of fusion tags to all target proteins to generate a platform 871 with synthesized or self-assembled peptides/proteins 870. The synthesized or self-assembled peptides/proteins 870 are then released LL to form the two and three-dimensional configurations 880.

In summary, disclosed here is a novel and unintuitive combinatory sequence of methods and approaches utilized in molecular biology and protein chemistry; in the non-obvious order arranged here, they enable experimentalists skilled in the art to obtain unlimited quantities of proteins from as little as a single peptide/protein template. Each step required has been demonstrated previously and is documented in the scientific literature. When assembled in the unintuitive way disclosed here, a new means of peptide/protein production results that will be of broad applicability and value to science, industry and national security, as it allows not only the mass production of specific peptides/proteins from a small number of templates but also enables the reproduction with high fidelity of post translational modifications on the molecule that give it specific characteristics and functions.

The invention has been described herein in considerable detail in order to comply with the Patent Statutes and to provide those skilled in the art with the information needed to apply the novel principles of the present invention, and to construct and use such exemplary and specialized components as are required. However, it is to be understood that the invention may be implemented by specifically different equipment, and devices, and that various modifications, both as to the equipment details and operating procedures, may be accomplished without departing from the true spirit and scope of the present invention.

### REFERENCES

The teachings of the following publications are incorporated herein by reference in their entirety.
1. Chang, Shuai; Huang, Shuo; Liu, Hao; et al.,“Chemical recognition and binding kinetics in a functionalized tunnel junction,” NANOTECHNOLOGY, Volume: 23 Issue: 23, Article Number: 235101 Published: JUN 15 2012.
2. By: He, Lin; Han, Xi; Ma, Bin, “De novo sequencing with limited number of post-translational modifications per peptide,” JOURNAL OF BIOINFORMATICS AND COMPUTATIONAL BIOLOGY, Volume: 11 Issue: 4, Article Number: UNSP 1350007, Published: AUG 2013.
3. Khorana H G, Agarwal K L, Büchi H et al. (December 1972). “Studies on polynucleotides. 103. Total synthesis of the structural gene for an alanine transfer ribonucleic acid from yeast”. J. Mol. Biol. 72 (2): 209-217. doi:10.1016/0022-2836(72)90146-5. PMID 4571075.
4. Itakura K, Hirose T, Crea R et al. “Expression in Escherichia coli of a chemically synthesized gene for the hormone somatostatin”. Science 198 (4321): 1056-1063 (December 1977). Bibcode: 1977Sci . . . 198.1056I. doi: 10.1126/science.412251.
5. Edge M D, Green A R, Heathcliffe G R et al. (August 1981). “Total synthesis of a human leukocyte interferon gene,” Nature 292 (5825): 756-62. Bibcode: 1981 Natur.292.756E. doi: 10.1038/292756a0. PMID 6167861.
6. Fuhrmann M, Oertel W, Hegemann P, “A synthetic gene coding for the green fluorescent protein (GFP) is a versatile reporter in Chlamydomonas reinhardtii”. Plant J. 19 (3): 353-61(August 1999). doi: 10.1046/j.1365-313X.1999.00526.x. PMID 10476082.
7. Mandecki W, Bolling T J (August 1988). “FokI method of gene synthesis”. Gene 68 (1): 101-7. doi: 10.1016/0378-1119(88)90603-8. PMID 3265397.
8. Stemmer W P, Crameri A, Ha K D, Brennan T M, Heyneker H L, “Single-step assembly of a gene and entire plasmid from large numbers of oligodeoxyribonucleotides,” Gene 164 (1): 49-53(October 1995). doi: 10.1016/0378-1119(95)00511-4. PMID 7590320.
9. (Wikipedia [http://en.wikipedia.org/wiki/Artificial_gene_synthesis].
10. Welch M, Govindarajan M, Ness J E, Villalobos A, Gurney A, Minshull J, Gustafsson C (2009). “Design Parameters to Control Synthetic Gene Expression in Escherichia coli”. In Kudla, Grzegorz. PLoS ONE 4 (9): e7002.
11. “Protein Expression”. DNA2.0. Retrieved 11 May 2010.
12. Protein microarrays as tools for functional proteomics By: LaBaer, J; Ramachandran, CURRENT OPINION IN CHEMICAL BIOLOGY, Volume: 9 Issue: 1 Pages: 14-19 Published: FEB 2005.
13. Ramachandran N, Hainsworth E, Bhullar B, Eisenstein S, Rosen B, Lau A Y, Walter J C, LaBaer J: “Self-assembling protein Microarrays,” Science 2004, 305:86-90.
14. Ramachandran, Niroshan; Srivastava, Sanjeeva; LaBaer, Joshua, “Applications of protein microarrays for biomarker discovery,” PROTEOMICS CLINICAL APPLICATIONS, Volume: 2 Issue: 10-11 Pages: 1444-1459 Published: OCT 2008.
15. Festa F¹, Rollins S M, Vattem K, Hathaway M, Lorenz P, Mendoza E A, Yu X, Qiu J, Kilmer G, Jensen P, Webb B, Ryan E T, LaBaer J., “Robust microarray production of freshly expressed proteins in a human milieu,” Proteomics Clin Appl. 2013 June; 7(5-6):372-7. doi: 10.1002/prca.201200063. Epub 2013 May 17.

What is claimed is:

1. A peptide/protein amplification reaction (PAR) method comprising:
selecting or capturing an amino acid chain of interest for amplification;
denaturing the amino acid chain of interest including at least one amino acid sequence;
reading the at least one amino acid sequence and any protein modifications present;
synthesizing at least one mRNA template, mRNA equivalent, or cDNA template complementary to the read amino acid sequence; and
translating the mRNA, mRNA equivalent, or cDNA to obtain an amplified amino acid chain of interest using a biological system.

2. The method of claim 1 wherein the amino acid chain of interest comprises a peptide or protein.

3. The method of claim 1 wherein the biological system comprises a ribosome driven system.

4. The method of claim 1 wherein the step of reading the at least one amino acid sequence and applicable protein modifications comprises processing the at least one amino acid sequence by operating a scanning tunneling microscope and/or an atomic force microscope.

5. The method of claim 1 wherein the step of reading the at least one amino acid sequence and applicable protein modifications comprises processing the at least one amino acid sequence by performing de novo sequencing using mass spectrometry.

6. The method of claim 1 wherein the step of synthesizing at least one mRNA template, mRNA equivalent, or cDNA template includes transcribing the read amino acid sequence to synthesize a mRNA, mRNA equivalent, or cDNA using oligonucleotide synthesis.

7. The method of claim 1 further comprising increasing the number of mRNA templates, mRNA equivalents, or cDNA.

8. The method of claim 7 wherein the step of increasing the number of mRNA equivalent templates comprises processing the mRNA template, mRNA equivalent, or cDNA template by nucleic acid amplification (PCR).

9. The method of claim 7 wherein the step of increasing the number of mRNA templates, mRNA equivalents, or cDNA comprises processing the mRNA template, mRNA equivalent, or cDNA by RT-PCR.

10. The method of claim 1 further comprising modifying the amplified amino acid chain of interest.

11. The method of claim 1 wherein the step of to obtain the amplified amino acid chain of interest using a biological system is carried out in vivo.

12. The method of claim 1 wherein the step to obtain the amplified amino acid chain of interest using a biological system is carried out in vitro.

13. The method of claim 1 wherein the step to obtain the amplified amino acid chain of interest using a biological system is carried out on a solid surface.

14. The method of claim 1 wherein the step to obtain the amplified amino acid chain of interest using a biological system is carried out on a solid surface with immobilized protein.

15. A peptide/protein amplification reaction (PAR) method comprising:
selecting or capturing an amino acid chain of interest for amplification, wherein the amino acid chain of interest comprises a peptide or protein;
denaturing the amino acid chain of interest including at least one amino acid sequence;
reading the at least one amino acid sequence;
transcribing the read amino acid sequence to synthesize at least one mRNA template, mRNA equivalent, or cDNA using oligonucleotide synthesis;
increasing the number of mRNA templates, mRNA equivalents, or cDNA equivalent templates; and
translating the mRNA, mRNA equivalent, or cDNA of interest to obtain a high copy number of the target amino acid sequence using a biological system.

16. The method of claim 15 wherein the biological system comprises a ribosome driven system.

17. The method of claim 15 wherein the step of reading the at least one amino acid sequence comprises processing the at least one amino acid sequence by operating a scanning tunneling microscope and/or an atomic force microscope.

18. The method of claim 15 wherein the step of increasing the number of mRNA templates, mRNA equivalents, or cDNA comprises processing the mRNA templates, mRNA equivalents, or cDNA by nucleic acid amplification (PCR).

19. The method of claim 15 wherein the step of increasing the number of mRNA templates, mRNA equivalents, or cDNA comprises processing the mRNA templates, mRNA equivalents, or cDNA by RT-PCR.

20. The method of claim 15 further comprising modifying the amplified amino acid chain of interest.

21. The method of claim 1 wherein the step to obtain the amino acid chain of interest using a biological system is carried out in vivo.

22. The method of claim 15 wherein the step to obtain the amino acid chain of interest using a biological system is carried out in vitro.

23. The method of claim 15 wherein the step to obtain the amino acid chain of interest using a biological system is carried out in vitro on a solid surface.

24. The method of claim 15 wherein the step to obtain the amino acid chain of interest using a biological system is carried out in vitro on a solid surface on which the resultant proteins have been immobilized.
